(19) United States
(12) Patent Application Publication
(10) Pub. No.: US 2022/0105211 A1
(43) Pub. Date: Apr. 7, 2022

(54) DISINFECTION DEVICE

(71) Applicant: SOCLEAN, INC., PETERBOROUGH, NH (US)

(72) Inventor: Janine Eisner-Wall, Peterborough, NH (US)

(73) Assignee: SOCLEAN, INC., PETERBOROUGH, NH (US)

(21) Appl. No.: 17/490,896

(22) Filed: Sep. 30, 2021

Related U.S. Application Data
(60) Provisional application No. 63/087,039, filed on Oct. 2, 2020.

(51) Int. Cl. A61L 2/10; A61L 2/26

(52) U.S. Cl. CPC A61L 2/10; A61L 2/26; A61L 2202/24; A61L 2202/122; A61L 2202/11

(57) ABSTRACT

A disinfection device may include a body having a mount configured to couple to a surface, a sanitization compartment defining a cavity having at least one open end, at least one light source configured emit sanitizing light into the cavity, and a lid configured to transition between an open position and a closed position, wherein, when the lid is in the closed position, the lid extends over the open end.

### TECHNICAL FIELD

The present disclosure is generally directed to disinfection devices and more specifically to a disinfection device configured to disinfect a device using a sanitizing light (e.g., ultraviolet light) that is incident on one or more surfaces of the device.

### BACKGROUND INFORMATION

Devices (e.g., medical devices, consumer devices, and etc.) are exposed to numerous environments in which harmful bacteria and/or viruses can come into contact with and reside on the devices. Without proper disinfection, further use of these devices after exposure can result in the spread of harmful bacteria and/or viruses. Accordingly, reusable devices may be disinfected before reuse in order to reduce the risk of spreading harmful bacteria and viruses.

### BRIEF DESCRIPTION OF THE DRAWINGS

These and other features advantages will be better understood by reading the following detailed description, taken together with the drawings wherein:

FIG. 1 shows a schematic example of a disinfection device, consistent with embodiments of the present disclosure.

FIG. 2 shows a perspective view of a disinfection device having a lid in a closed position, consistent with embodiments of the present disclosure.

FIG. 3 shows a perspective view of the disinfection device of FIG. 2 having the lid in an open position, consistent with embodiments of the present disclosure.

FIG. 4 shows a cross-sectional view of the disinfection device of FIG. 2 having the lid in an open position, consistent with embodiments of the present disclosure.

FIG. 5 shows a front view of the disinfection device of FIG. 2, consistent with embodiments of the present disclosure.

FIG. 6 shows a perspective view of a disinfection device having a lid in an open position, consistent with embodiments of the present disclosure.

FIG. 7 shows a side view of the disinfection device of FIG. 6 having the lid in a closed position, consistent with embodiments of the present disclosure.

FIG. 8 shows a bottom perspective view of the disinfection device of FIG. 6, consistent with embodiments of the present disclosure.

FIG. 9 shows a cross-sectional perspective view of the disinfection device of FIG. 6, consistent with embodiments of the present disclosure.

FIG. 10 shows a magnified view of an example of a device within the disinfection device of FIG. 6, consistent with embodiments of the present disclosure.

FIG. 11 shows a front view of a disinfection device, consistent with embodiments of the present disclosure.

FIG. 12 shows a side view of the disinfection device of FIG. 11, wherein a portion of the disinfection device is shown as transparent for purposes of clarity, consistent with embodiments of the present disclosure.

FIG. 13 shows a front view of a disinfection device, consistent with embodiments of the present disclosure.

FIG. 14 shows a front view of a disinfection system that includes the disinfection devices of FIGS. 12 and 13, consistent with embodiments of the present disclosure.

FIG. 15 shows a perspective view of a disinfection system, consistent with embodiments of the present disclosure.

FIG. 16 shows a perspective view of a sanitization platform of the disinfection system of FIG. 15, consistent with embodiments of the present disclosure.

FIG. 17 shows a perspective view of a partially assembled disinfection system, consistent with embodiments of the present disclosure.

FIG. 18 shows a side view of a disinfection system, consistent with embodiments of the present disclosure.

FIG. 19 shows a schematic view of a light source, consistent with embodiments of the present disclosure.

FIG. 20 shows a schematic view of a light source, consistent with embodiments of the present disclosure.

### DETAILED DESCRIPTION

The present disclosure is generally directed to a disinfection device configured to generate a sanitizing light (e.g., ultraviolet light such as ultraviolet C light). The disinfection device includes one or more sidewalls that define a sanitization chamber, a lid, and a light source. The sanitization chamber defines a cavity having at least one open end, wherein the lid is configured to selectively extend across the open end, enclosing the cavity. At least a portion of one or more surfaces defining the enclosed cavity may be at least partially reflective such that light generated by the light source is at least partially reflected therefrom. The sanitization chamber may further include a light transmissive portion such that light emitted into the cavity by the light source can pass therethrough. As such, the light transmissive portion can provide a visible indication that the light source is active.

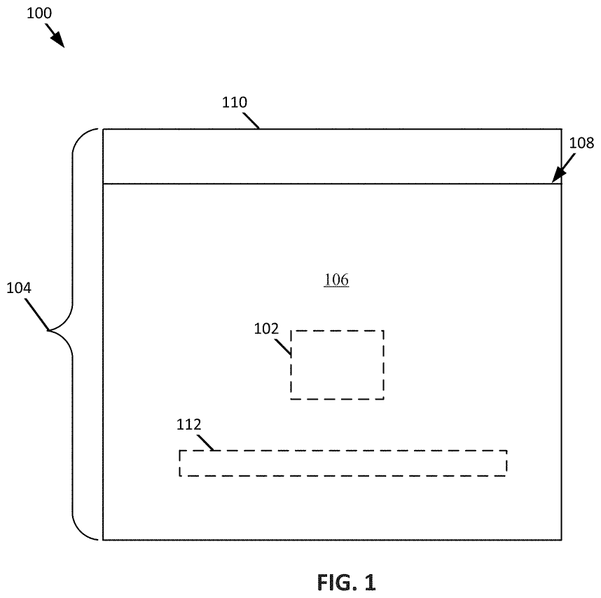

FIG. 1 shows a schematic example of a disinfection device 100 configured to disinfect a device 102 (e.g., a consumer electronic device such as a mobile phone, laptop, tablet, or the like). In some instances, the disinfection device 102 may be further configured to charge one or more batteries included within the device 102 (e.g., using a wired or wireless charging system).

As shown, the disinfection device 100 includes a sanitization compartment 104 defining a cavity 106 having an open end 108, a lid 110 configured to extend over the open end 108 and selectively enclose the cavity 106, and a light source 112 (e.g., a light emitting diode, a low pressure lamp, a high pressure lamp, and/or any other light source) configured to generate sanitizing light (e.g., ultraviolet C light). Light generated by the light source 112 is incident on the device 102 and/or one or more sidewalls defining the enclosed cavity 106. For example, at least a portion of the emitted light may be directly incident on the device 102 and at least a portion of the emitted light may be indirectly incident (e.g., light that is reflected from the one or more sidewalls) on the device 102. Accordingly, at least a portion of the one or more sidewalls defining the enclosed cavity may be at least partially reflective to the emitted light (e.g., at least 50%, at least 60%, at least 70%, at least 80%, at least 85%, at least 90%, or at least 95% of the emitted light is reflected). For example, a surface of the lid 110 that defines a portion of the enclosed cavity may be at least partially reflective to the emitted light.

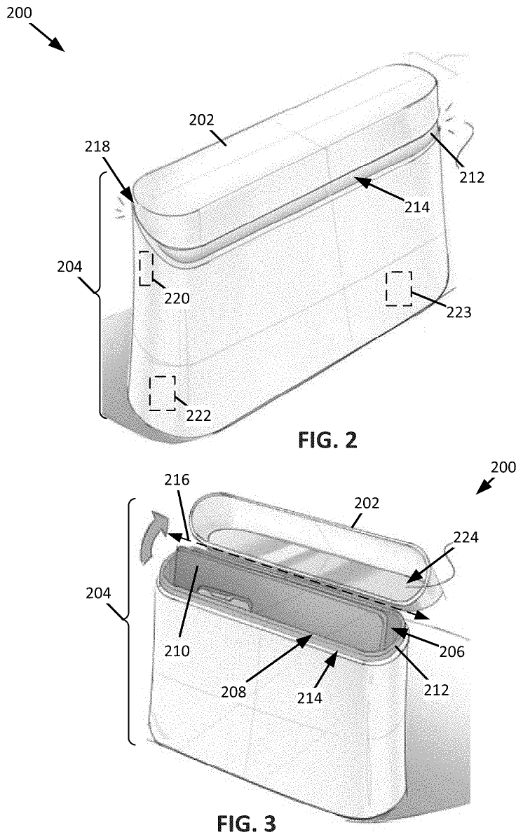

FIG. 2 shows a perspective view of a disinfection device 200 having a lid 202 in a closed position and FIG. 3 shows a perspective view of the disinfection device 200 having the lid 202 in an open position, wherein the disinfection device 200 may be an example of the disinfection device 100 of FIG. 1. As shown, the disinfection device 200 includes a sanitization compartment 204 defining cavity 206 having an open end 208, wherein the lid 202 is configured to extend over the open end 208 to selectively enclose the cavity 206. The open end 208 is configured to receive a device 210 such that the device 210 can be positioned within the cavity 206.

As shown, a light diffuser 212 can extend around at least a portion of the cavity 206 (e.g., define at least a portion of the open end 208 of the cavity 206). The light diffuser 212 is configured to be light transmissive such that light incident thereon passes therethrough. In some instances, the light diffuser 212 may be configured to diffuse light passing therethrough. When the lid 202 is in the closed position, the lid 202 may engage (e.g., contact) the light diffuser 212 such that at least one emission surface 214 of the light diffuser 212 is exposed to the surrounding environment. As such, in operation, at least a portion of the light generated within the sanitization compartment 204 passes through the light diffuser 212 and is emitted from the emission surface 214, providing an indication of operation status to a user of the disinfection device 200.

The lid 202 can be configured to pivot between the open and the closed position about a pivot axis 216 of a hinge 218. The hinge 218 may include one or more biasing mechanisms (e.g., one or more springs) configured to urge the lid 202 towards the open or closed position. In some instances, the lid 202 may have sufficient mass to overcome the biasing force of the one or more biasing mechanisms after the lid 202 has been transitioned a predetermined distance towards one of the open position or closed position. In some instances, the lid 202 can further be configured to actuate a switch 220 when transitioning between the open and closed positions. For example, when transitioning towards the closed position, the lid 202 can actuate the switch 220 and cause a disinfection process to begin and, when transitioning to the open position, the lid 202 can actuate the switch 220 and cause the disinfection process to end (if the disinfection process in ongoing). In some instances, actuation of the switch may result in a corresponding activation of a timing circuit 222, the timing circuit 222 being configured to cause sanitizing light to be generated for a predetermined period of time. Additionally, or alternatively, the disinfection device 200 may include a controller 223 configured to cause one or more instructions to be carried out. The instructions may include sanitization profiles (e.g., corresponding to a time period along which a disinfection process is carried), safety profiles (e.g., corresponding to premature termination of a disinfection process such as in response to an opening of the lid 202), and/or any other operation profile.

At least a portion of a cavity facing surface 224 of the lid 202 can be at least partially reflective (e.g., at least 50%, at least 60%, at least 70%, at least 80%, at least 85%, at least 90%, or at least 95% of light incident thereon is reflected). Such a configuration may increase an exposure of the device 210 to sanitizing light generated within the sanitization compartment 204.

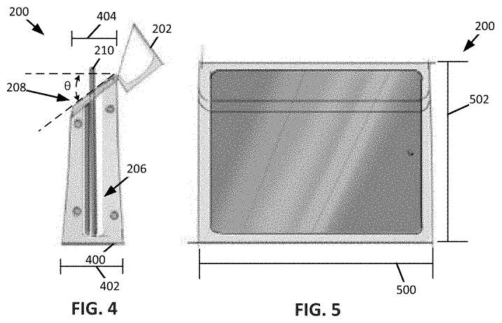

FIG. 4 shows a cross-sectional view of the disinfection device 200 having the lid 202 in the open position. As shown, the open end 208 of the cavity 206 is angled according to an opening angle θ. The opening angle θ can generally be described as extending between a plane that extends parallel to the open end 208 and a plane that extends parallel to a base 400 of the disinfection device 200 (e.g., a horizontal plane). The opening angle θ can be configured to allow easier access to the device 210 within the sanitization compartment 204. For example, a first portion of the open end 208 may be disposed below an opposing portion of the open end 208 such that the device 200 may extend above the first portion but not the second portion. The opening angle θ may measure, for example, between 45° and 75°. By way of further example, the opening angle θ may measure 60°. The lid 202 may have a lid angle α that corresponds to the opening angle θ such that the lid 202 engages (e.g., contacts) the light diffuser 212 when in the closed position, enclosing the open end 208 of the cavity 206.

A disinfection device base width 402 may measure, for example, in a range of 5 centimeters (cm) to 25 cm. By way of further example, the disinfection device base width 402 may measure 10.16 cm. A disinfection device top width 404 may measure, for example, in a range of 4 cm to 12 cm. By way of further example, the disinfection device top width 404 may measure 7.62 cm. As such, in some instances, the disinfection device 200 may generally be described as decreasing in the width with increasing distance from the base 400.

FIG. 5 shows a front view of an example of the disinfection device 200. As shown, in this example, the disinfection device 200 may have a disinfection device length 500 and a disinfection device height 502. The disinfection device height 502 may measure, for example, in a range of 5 cm to 45 cm and the disinfection device length 500 may measure, for example, in a range of 15 cm to 50 cm. By way of further example, the disinfection device height 502 may measure, for example, 30.48 cm and the disinfection device length 500 may measure, for example, 38.1 cm. By way of still further example, the disinfection device height 502 may measure, for example, 11.43 cm and the disinfection device length 500 may measure, for example, 20.32 cm. In some instances, a ratio of disinfection device height 502 to the disinfection device length 500 (i.e., the disinfection device height 502 divided by the disinfection device length 500) may be, for example, 4/5. By way of further example, the ratio of the disinfection device height 502 to the disinfection device length 500 may be 9/16.

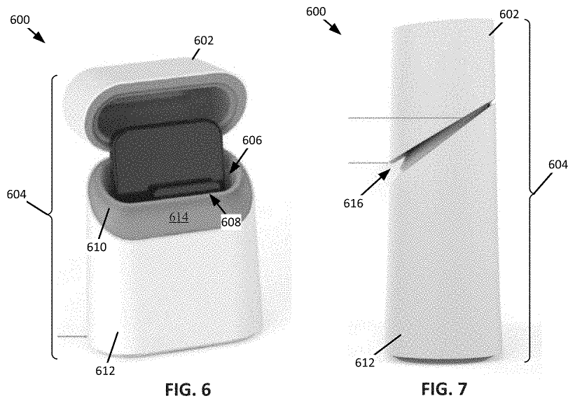

FIG. 6 shows a perspective view of a disinfection device 600 having a lid 602 in an open position and FIG. 7 shows a side view of the disinfection device 600 having the lid 602 in a closed position, wherein the disinfection device 600 may be an example of the disinfection device 200 of FIG. 2. As shown, the disinfection device 600 includes a sanitization compartment 604 defining a cavity 606 having an open end 608, wherein the lid 602 is configured to selectively extend across the open end 608, enclosing the cavity 606. A light diffuser 610 may extend around at least a portion of the cavity 606 (e.g., define at least a portion of the open end 608). The light diffuser 610 is configured to allow light generated within the cavity 606 to be emitted into a surrounding environment. As such, the light diffuser 610 may generally be described as being configured to provide an indication that a disinfection process is in process. The light diffuser 610 may diffuse light passing therethrough.

As shown in FIG. 7, when in the closed position, at least a portion of the lid 602 is vertically spaced apart from a body 612 of the disinfection device 600 such that a portion of the lid 602 obscures a portion of an emission surface 614 of the light diffuser 610. The portion of the lid 602 obscuring the emission surface 614 may be horizontally spaced apart from the emission surface 614. As such, a gap 616 can extend between the lid 602, the body 612, and the emission surface 614. The gap 616 can be sized to receive, for example, a finger therein. As such, the gap 616 can generally be described as being configured to facilitate the transitioning of the lid 602 from the closed position to the open position.

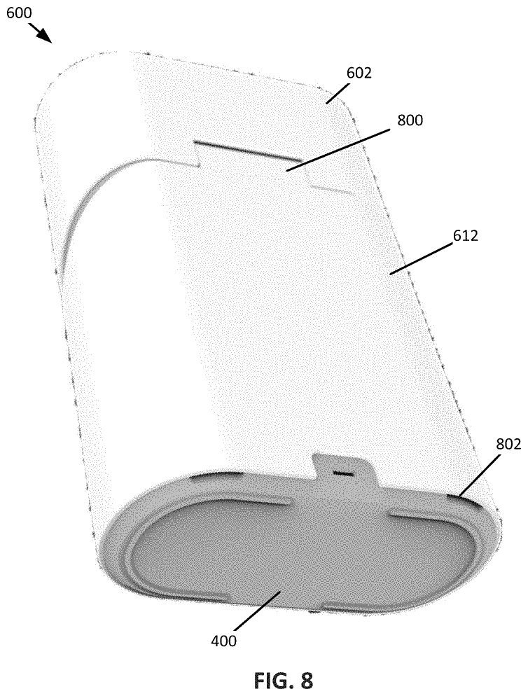

FIG. 8 is a bottom perspective view of the disinfection device 600 of FIG. 6. As shown, the lid 602 can be pivotally coupled to the body 612 using a hinge 800. As also shown, the base 400 can include one or more vents 802. The one or more vents 802 may provide airflow to one or more components (e.g., a light source) of the disinfection device 600.

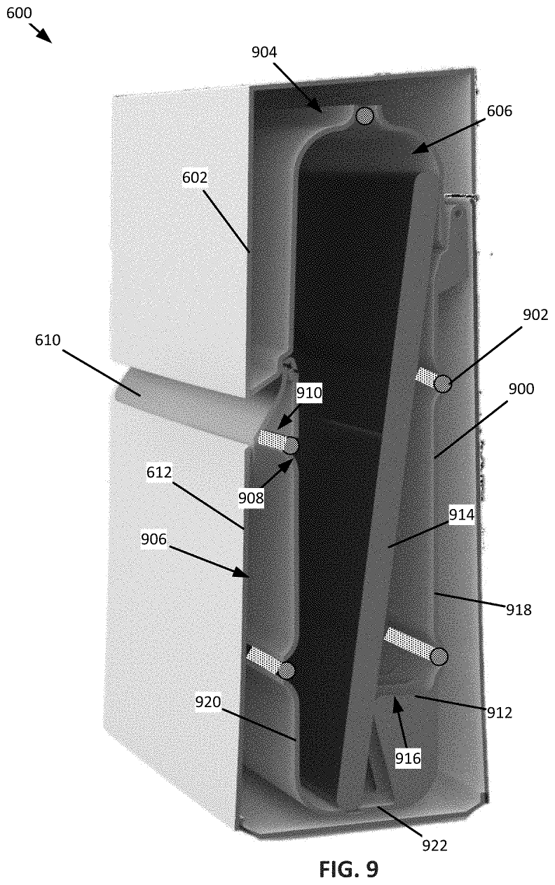

FIG. 9 shows a cross-sectional perspective view of the disinfection device 600 of FIG. 6. As shown, the disinfection device 600 includes a sanitization frame 900 that extends within the body 612, defining at least a portion of the cavity 606. In some instances, the lid 602 may include at least a portion of the sanitization frame 900. In these instances, when the lid 602 is in the closed position, the cavity 606 extends into the lid 602.

The sanitization frame 900 can be configured to receive one or more light sources 902 within respective receptacles 904 defined within the sanitization frame 900. The one or more light sources 902 are configured to generate sanitizing light (e.g., ultraviolet C light). At least one of the one or more light sources 902 can be configured to emit sanitizing light both into the cavity 606 and into the light diffuser 610. For example, at least one of the one or more light sources 902 can be configured to emit omni-directional light such that at least a portion of the generated light is emitted into the cavity 606 and at least a portion of the generated light is incident on the light diffuser 610.

For example, and as shown, an internal pathway 906 may extend between the sanitization frame 900 and the body 612 of the disinfection device 600. The internal pathway 906 can be configured to allow a portion of the light emitted from the one or more light sources 902 to be incident on the light diffuser 610. For example, and as shown, at least one of the receptacles 904 may include a first emission opening 908 that opens into the cavity 606 and a second emission opening 910 that opens into the internal pathway 906. As such, for example, when a light source 902 that is configured to generate omni-directional light is positioned within the receptacle 904, light can be emitted through both the first emission opening 908 and the second emission opening 910.

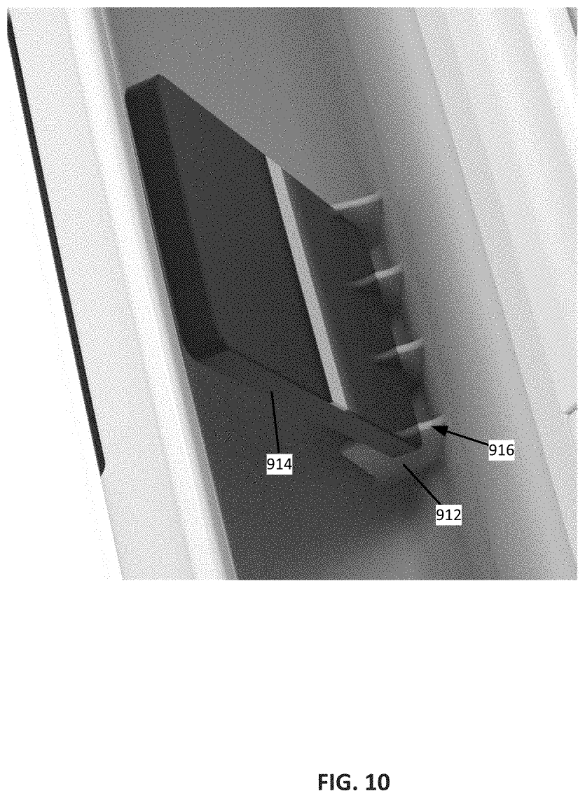

As shown, the sanitization frame 900 may also include a plurality of standoffs 912. The standoffs 912 may be configured to support a device 914 on a support surface 916 (e.g., as shown in FIG. 10 in which a smaller example of the device 914 is shown positioned on the standoffs). As such, the standoffs 912 may improve an accessibility of the device 914 within the sanitization cavity 606. The standoffs 912 may be spaced apart from each other such that light can pass into the space defined between the standoffs 912. Additionally, or alternatively, the standoffs 912 may be substantially transparent to the sanitizing light within the cavity 606 (e.g., at least 50%, at least 75%, at least 85%, at least 90%, or at least 95% of sanitizing light incident thereon passes therethrough). When not supporting the device 914 on the support surface 916, standoffs 912 may be configured to orient the device 914 within the sanitization cavity 606 such that the device 914 does not interfere with the lid 602 when the lid 602 transitions between the open and closed positions. For example, the standoffs 912 may be configured such that the device 914 extends from a first side 918 of the sanitization frame 900 towards a second, opposing, side 920 of the sanitization frame 900, wherein the standoffs 912 extend from the second side 920 in a direction of the first side 918 and along a base 922 of the sanitization frame 900, the base 922 extending between the first and second sides 918 and 920.

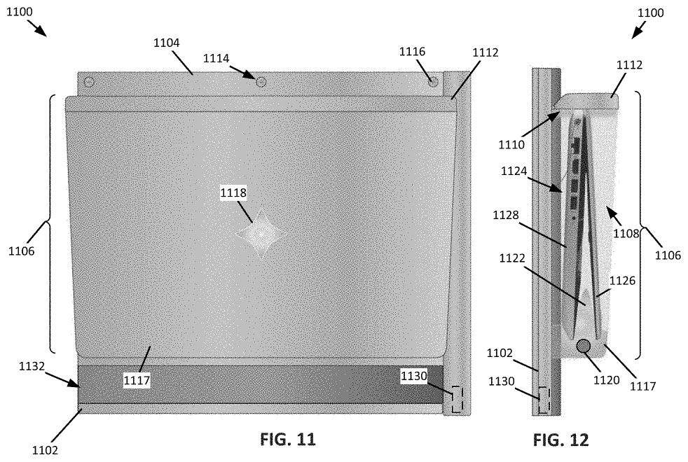

FIG. 11 shows a front view of a disinfection device 1100 and FIG. 12 shows a side view of the disinfection device 1100, wherein the disinfection device 1100 may be an example of the disinfection device 100 of FIG. 1. The disinfection device 1100 includes a body 1102 having a mount 1104, a sanitization compartment 1106 that defines a cavity 1108 having at least one open end 1110, and a lid 1112 configured to transition between an open and a closed position, wherein, when in the closed position, the lid 1112 extends over the open end 1110 and defines a portion of the cavity 1108. The mount 1104 is configured to couple the disinfection device 1100 to a surface extending adjacent to the mount 1104 (e.g., a wall). The mount 1104 can include one or more apertures 1114 configured to receive one or more mounting fixtures 1116 (e.g., a screw, nail, and/or any other mounting fixture) configured to couple the disinfection device 1100 to a surface extending adjacent to the mount 1104.

As shown, the sanitization compartment 1106 may include one or more sidewalls 1117 that collectively define at least a portion of the cavity 1108. At least one of the one or more sidewalls 1117 may include a light diffuser 1118. The light diffuser 1118 may include a light transparent material and may be configured to allow light emitted into the cavity 1108 to pass through the light diffuser 1118. The light diffuser 1118 may be configured to diffuse light passing therethrough. As such, the light diffuser 1118 may generally be described as being configured to indicate an operational status (i.e., on or off) of one or more light sources 1120 that are configured to emit sanitizing light (e.g., ultraviolet C light) into the cavity 1108.

A standoff 1122 may be disposed within the cavity 1108. For example, the standoff 1122 can extend along the lid 1112 such that, when the lid 1112 is in the closed position, the standoff 1122 is positioned within the cavity or along a surface that is generally opposite the lid 1112 when the lid 1112 is in the closed position (e.g., a base surface). The standoff 1122 is configured to orient and/or support a device 1124 within the cavity 1108 such that light emitted into the cavity 1108 by the light source 1120 is incident on multiple surfaces of the device 1124. For example, and as shown, when the device 1124 is a foldable device (e.g., a laptop computer), the device 1124 includes a first portion 1126 pivotally coupled to a second portion 1128, wherein the standoff 1122 extends between the first and second portions 1126 and 1128 (e.g., at a location opposite the pivot point of the first and second portions 1126 and 1128). As such, the standoff 1122 can generally be described as separating the first portion 1126 from the second portion 1128 when the device is received within the cavity 1108. Such a configuration allows light to extend between the first and second portions 1126 and 1128, sanitizing surfaces of the first and second portions 1126 and 1128 that face each other. For example, when the first portion 1126 includes a screen and the second portion 1128 includes a keyboard that faces the screen when the device 1124 is in a folded position, the screen and keyboard may be disinfected by light extending between the first and second portions 1126 and 1128. The standoff 1122 may be transparent to the light emitted by the one or more light sources 1120. For example, the standoff 1122 may include at least one of the one or more light sources 1120.

As shown, the body 1102 may include at least one power coupling 1130. The power coupling 1130 can be configured to directly electrically couple a power supply (e.g., a power outlet or one or more batteries). In some instances, the power coupling 1130 can be configured to electrically couple to a second disinfection device such that a disinfection system comprising at least two disinfection devices can be formed, wherein the disinfection devices share a common power supply.

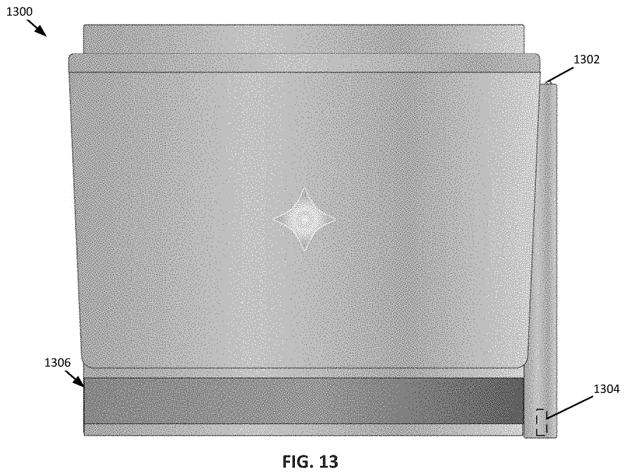

FIG. 13 shows an example of a disinfection device 1300 configured to electrically couple to the power coupling 1130 of the disinfection device 1100 of FIG. 11. In this context, when the disinfection device 1100 only has a single power coupling 1130 and is configured to electrically couple to the disinfection device 1300, the disinfection device 1100 may generally be referred to as a head disinfection device. As shown, the disinfection device 1300 includes a first power coupling 1302 configured to electrically couple to the power coupling 1130 of the head disinfection device 1100 and a second power coupling 1304 configured to electrically couple to a power source (e.g., directly or through one or more additional disinfection devices). As such, the disinfection device 1300 may be generally referred to as a bridging disinfection device. In some instances, the second power coupling 1304 may be electrically coupled to one or more additional bridging disinfection devices. For example, an additional bridging disinfection device may physically couple to the bridging disinfection device 1300 using a coupling track 1306. In some instances, the second power coupling 1304 may be directly electrically coupled to a power source (e.g., one or more batteries or a power outlet). In some instances, the bridging disinfection device 1300 may physically couple to the head disinfection device 1100 using a coupling track 1132. Additionally, or alternatively, the bridging disinfection device 1300 may physically couple to the head disinfection device 1100 using one or more of one or more magnets, one or more snap-fits, one or more friction (or press) fits, one or more adhesives, one or more threaded fasteners, and/or any other suitable form of coupling. In some instances, the coupling can be configured such that the bridging disinfection device 1300 can be removed from the head disinfection device 1100 such that a different bridging disinfection device can be coupled to the head disinfection device 1100. As such, a resulting disinfection system comprising the head disinfection device 1100 and the bridging disinfection device 1300 may generally be described as being modular.

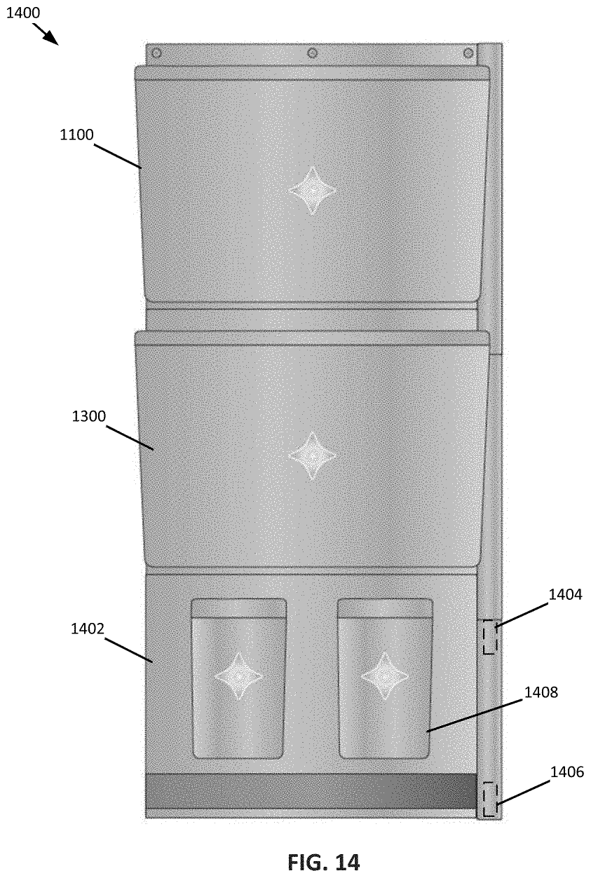

FIG. 14 shows an example of a disinfection system 1400 that includes the head disinfection device 1100, a tail disinfection device 1402, and one or more bridging disinfection devices 1300. The one or more bridging disinfection devices 1300 are electrically coupled to the head disinfection device 1100 and the tail disinfection device 1402 such that power can be provided from the tail disinfection device 1402 to the head disinfection device 1100 via the one or more bridging disinfection devices 1300. The tail disinfection device 1402 can have a first power coupling 1404 configured to electrically couple to a respective bridge disinfection device 1300 and a second power coupling 1406 configured to directly electrically couple to a power source (e.g., one or more batteries or a power outlet).

Each of the disinfection devices 1100, 1300, and 1402 may be physically coupled together using, for example, coupling tracks 1132 and 1306. Additionally, or alternatively, the disinfection devices 1100, 1300, and 1402 may be physically coupled together using any one or more of one or more magnets, one or more snap-fits, one or more friction (or press) fits, one or more adhesives, one or more threaded fasteners, and/or any other suitable form of physical coupling. In some instances, the coupling can be configured such that the disinfection devices 1100, 1300, and 1402 forming the disinfection system 1400 can be interchangeable with one or more additional disinfection devices. As such, the disinfection system 1400 may generally be described as being modular.

As shown, the tail disinfection device 1402 may include a plurality of sanitization compartments 1408, wherein at least one sanitization compartment 1408 has a size different from that of at least one other sanitization compartment 1408. For example, at least one sanitization compartment 1408 may correspond to a mobile phone and at least one sanitization compartment 1408 may correspond to a laptop computer. Similarly, one or more of the head and/or bridging disinfection devices 1100 and 1300 may include a plurality of sanitization compartments, which may be sized for various devices. In instances where the disinfection devices 1100, 1300, and 1402 include multiple sanitization compartments, the sanitization compartments may share at least one light source.

In some instances, the tail disinfection device 1402 may include a support configured to support the disinfection system 1400 on a surface (e.g., a floor). In these instances, the mount 1104 of the head disinfection device 1100 may not be coupled to a surface (e.g., a wall) that extends generally perpendicular to the surface on which the support is positioned.

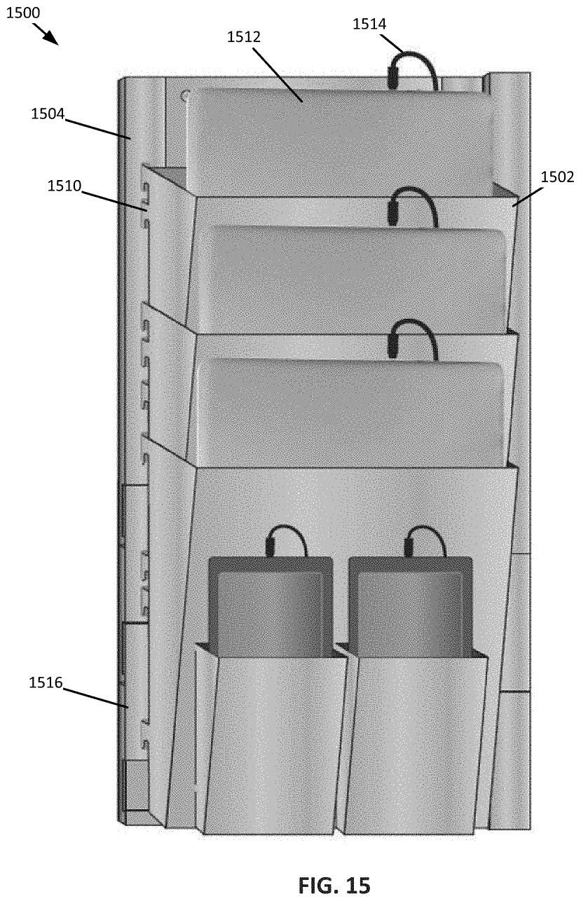

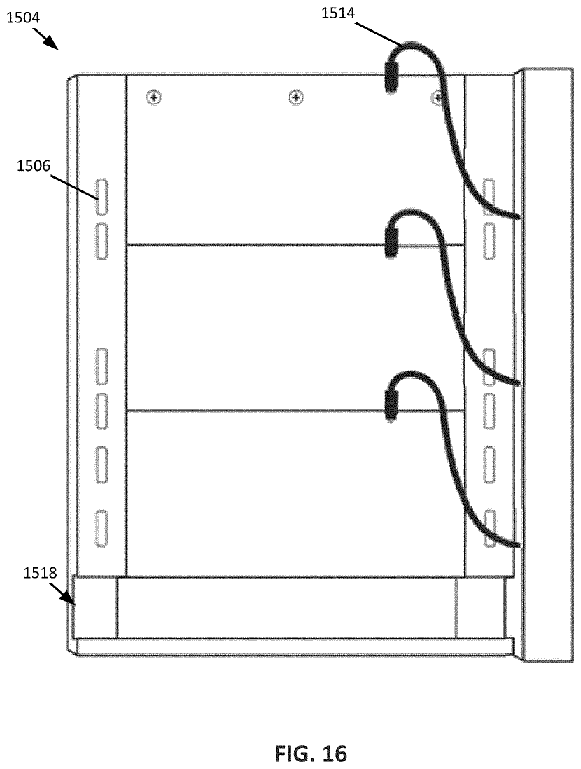

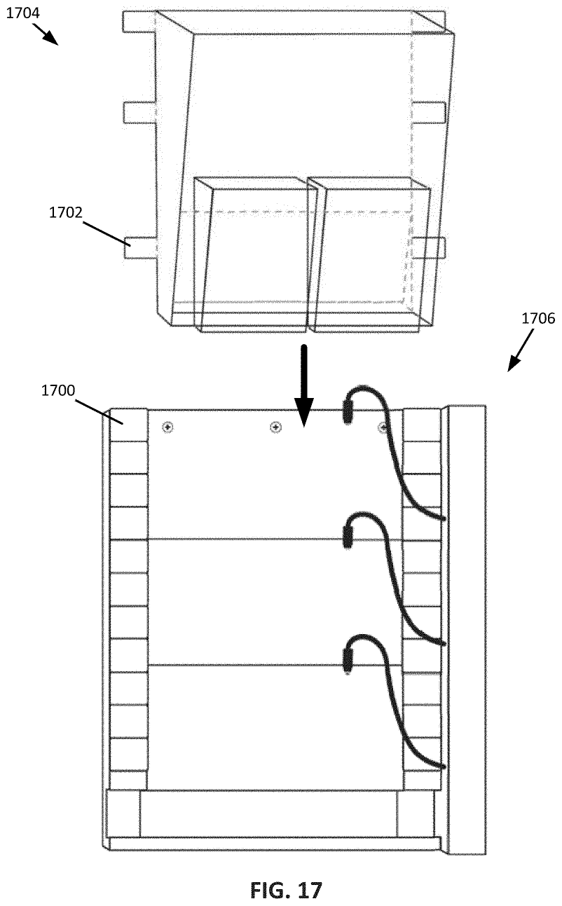

FIG. 15 shows an example of a disinfection system 1500 including one or more disinfection devices 1502, which may be examples of the disinfection device 100 of FIG. 1, removably coupled to a sanitization platform 1504. FIG. 16 shows an example of the sanitization platform 1504. The sanitization platform 1504 includes a plurality of mounting points 1506. The mounting points 1506 are configured to couple to one or more disinfection devices 1502. For example, the one or more disinfection devices 1502 may include one or more hooks 1510 configured to couple to the sanitization platform 1504 at the mounting points 1506. For example, as shown, the mounting points 1506 may define slots configured to receive the hooks 1510. In some instances, the mounting points 1506 may include a power coupling configured to supply power to a corresponding disinfection device 1502. By way of further example, as shown in FIG. 17, one or more mounting points 1700 may define slots configured to slideably receive tabs 1702 extending from the disinfection device 1704 such that the disinfection device 1704 is coupled to a sanitization platform 1706 in response to the tabs 1702 being slideably received within the mounting points 1700. When the disinfection device 1704 is physically coupled to the sanitization platform 1706, the tabs 1702 may be configured to electrically couple with the mounting points 1700 such that electrical power can be delivered to the disinfection device 1704. Additionally, or alternatively, the one or more disinfection devices 1502 may be coupled to the sanitization platform 1504 at the mounting points 1506 using one or more of one or more magnets, one or more snap-fits, one or more friction (or press) fits, one or more adhesives, one or more threaded fasteners, and/or any other suitable form of coupling. Such a configuration may allow for disinfection devices of different sizes and/or configurations to be positioned in different locations along the sanitization platform 1504, allowing for the disinfection system 1500 to be configured by a user in order to meet specific requirements of the user.

The sanitization platform 1504 may be configured to supply power to one or more of the one or more disinfection devices 1502 and/or one or more devices 1512 received by the disinfection device 1502. For example, the sanitization platform 1504 (or the one or more disinfection devices 1502) may include one or more charging cables 1514 configured to electrically couple to a corresponding one of the one or more devices 1512. Additionally, or alternatively, the one or more disinfection devices 1502 may be configured to wirelessly charge a corresponding device 1512 received therein.

As shown, a size of the sanitization platform 1504 may be increased using one or more platform expanders 1516. The one or more platform expanders 1516 may include power couplings configured to electrically couple the one or more platform expanders 1516 to the sanitization platform 1504 such that power can be shared therebetween. By increasing a size of the sanitization platform 1504, additional disinfection devices 1502 may be added to the disinfection system 1500. The one or more platform expanders 1516 may be physically coupled to the platform using a coupling track 1518 defined in the sanitization platform 1504. When the disinfection system 1500 includes a plurality of platform expanders 1516, one or more of the platform expanders 1516 may include a coupling track. Additionally, or alternatively, the sanitization platform 1504 and the one or more platform expanders 1516 may be physically coupled together using any one or more of one or more magnets, one or more snap-fits, one or more friction (or press) fits, one or more adhesives, one or more threaded fasteners, and/or any other suitable form of physical coupling.

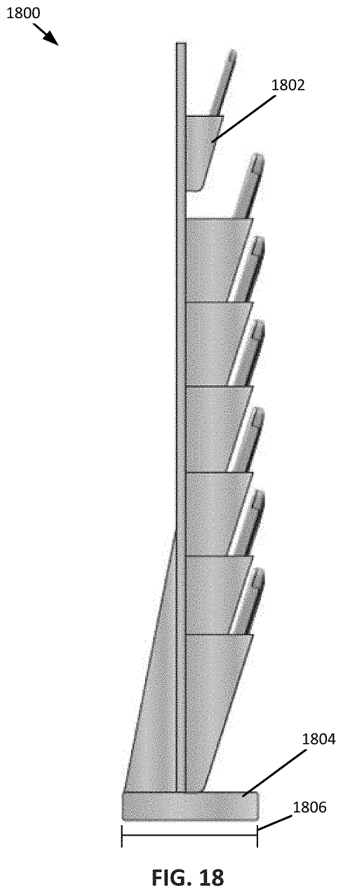

FIG. 18 shows a side view of a disinfection system 1800 having a plurality of disinfection devices 1802, which may be examples of the disinfection device 100 of FIG. 1. As shown, the disinfection system 1800 includes a support 1804 configured to support the disinfection system 1800 on a surface (e.g., a floor). A support width 1806 may be a maximum width of the disinfection system 1800. Such a configuration may increase the stability of the disinfection system 1800.

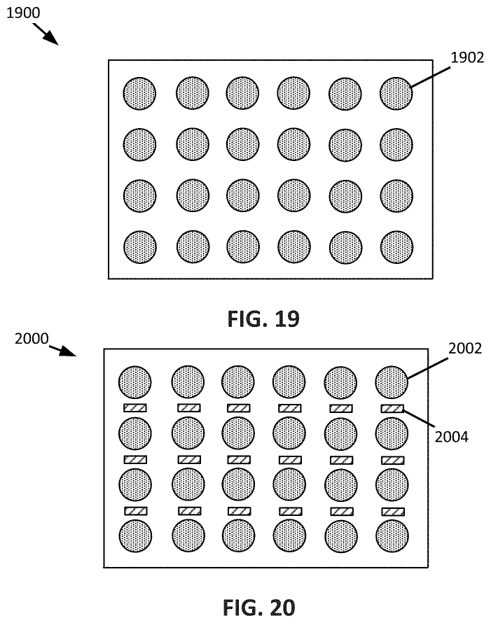

FIG. 19 shows a schematic example of a light source 1900, which may be an example of the light source 112 of the FIG. 1. As shown, the light source 1900 may include a plurality of light emitters 1902 (e.g., light emitting diodes). The light emitters 1902 can be configured to emit ultraviolet C light.

FIG. 20 shows a schematic example of a light source 2000, which may be an example of the light source 1900 of FIG. 19. As shown, the light source 2000 may include a plurality of light emitters 2002 (e.g., light emitting diodes configured to emit ultraviolet C light) and a plurality of light detectors 2004. The light emitters 2002 and light detectors 2004 can be arranged in an array. In operation, a location of a device to be disinfected, relative to the light source 2000 may be determined by pulsing the light sources 2000 (e.g., in a sequential order) and detecting (e.g., an intensity and/or a time of flight of) reflected light using the light detectors 2004. The location of the device to be disinfected, relative to the light source 2000, may be determined, for example, based, at least in part, on a detected change in intensity and/or time of flight. In other words, a location of a device to be disinfected relative to the light source 2000 may be determined based, at least in part, on one or more signals generated by the light detectors 2004.

Detection of a location of the device to be disinfected relative to the light source 2000 may allow selective activation of light emitters 2002 to minimize power consumption while maintaining a desired sanitization performance. In some instances, when disposed within a sanitization compartment, the device to be disinfected may be oriented between two or more of the light sources 2000. When two or more light sources 2000 are used and are oriented to emit light in opposing directions, the light detectors 2004 on an opposing light source 2000 may, additionally (or alternatively), be used in the determination of the location of the device relative to the light sources 2000.

In some instances, when the light source 2000 is used in a disinfection system where multiple disinfection devices may be available and/or in a modular system (e.g., the disinfection system 1400, 1500, 1800), the location of the device to be disinfected relative to the light source 2000 may be used to determine recommended configurations of the disinfection system (e.g., to best accommodate the device to be disinfected) and/or of the disinfection devices that are best sized for the device to be disinfected. The recommended configuration and/or disinfection device may be determined using, for example, a controller and provided to a user of the disinfection system.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the claims.

What is claimed is:

1. A disinfection device comprising:
a body having a mount configured to couple to a surface;
a sanitization compartment defining a cavity having at least one open end;
at least one light source configured emit sanitizing light into the cavity; and
a lid configured to transition between an open position and a closed position, wherein, when the lid is in the closed position, the lid extends over the open end.

2. The disinfection device of claim 1, wherein the mount includes at least one aperture configured to receive a mounting fixture.

3. The disinfection device of claim 2, wherein the mounting fixture is a screw.

4. The disinfection device of claim 1, wherein a standoff is disposed within the cavity.

5. The disinfection device of claim 4, wherein the standoff is transparent to light emitted by the at least one light source.

6. The disinfection device of claim 5, wherein the standoff includes the at least one light source.

7. The disinfection device of claim 4, wherein the standoff extends along the lid.

8. The disinfection device of claim 4, wherein the standoff extends along a surface opposite the lid.

9. The disinfection device of claim 1 further comprising a first power coupling and a second power coupling.

10. The disinfection device of claim 9, wherein the second power coupling is configured to directly electrically couple to a power supply.

11. The disinfection device of claim 10, wherein the first power coupling is configured to electrically couple to an additional disinfection device.

12. The disinfection device of claim 1, wherein the light source includes at least one light emitting diode configured to emit ultraviolet C light.

13. The disinfection device of claim 12, wherein the light source includes a plurality of light emitting diodes and a plurality of light detectors arranged in an array such that a position of a device to be disinfected relative to the light source can be determined based, at least in part, on one or more signals generated by the light detectors.

14. A disinfection system comprising:
a head disinfection device;
a tail disinfection device being configured to directly electrically couple to a power supply; and
a bridging disinfection device being configured to electrically couple to the head disinfection device and the tail disinfection device.

15. The disinfection system of claim 14, wherein the disinfection system includes a support.

16. The disinfection system of claim 15, wherein a support width is a maximum width of the disinfection system

17. The disinfection system of claim 15, wherein the head disinfection device includes a mount.

18. The disinfection system of claim 17, wherein the mount includes at least one aperture configured to receive a mounting fixture.

19. A disinfection system comprising:
a head disinfection device; and
a bridging disinfection device, the bridging disinfection device being configured to electrically couple to the head disinfection device and being configured to electrically couple to a power supply.

20. The disinfection system of claim 19, wherein the head disinfection device includes a mount having at least one aperture configured to receive a mounting fixture.
